(19) United States
(12) Patent Application Publication
(10) Pub. No.: US 2025/0172559 A1
(43) Pub. Date: May 29, 2025

(54) USE OF CD38+HLA-DR+CD8+ T CELL IN EARLY DIAGNOSIS OF ACUTE GRAFT-VERSUS-HOST DISEASE

(71) Applicant: Guangzhou First People's Hospital (Guangzhou Digestive Disease Center; Guangzhou First People's Hosp, Guangzhou (CN)

(72) Inventors: Yufeng Liu, Guangzhou (CN); Jie Cao, Guangzhou (CN); Limei Zhong, Guangzhou (CN); Yinchun Chen, Guangzhou (CN); Ruiqing Zhou, Guangzhou (CN); Shunqing Wang, Guangzhou (CN); Wenjian Mo, Guangzhou (CN)

(21) Appl. No.: 19/031,319

(22) Filed: Jan. 17, 2025

Related U.S. Application Data
(63) Continuation-in-part of application No. PCT/CN2023/116933, filed on Sep. 5, 2023.

(30) Foreign Application Priority Data
Sep. 5, 2022 (CN) 202211078691.8

(51) Int. Cl. G01N 33/569

(52) U.S. Cl. CPC G01N 33/56972; G01N 2333/70517; G01N 2333/70539; G01N 2333/70596

(57) ABSTRACT

Provided are a detection reagent for acute graft-versus-host disease (aGVHD) and the use of the percentage of CD38⁺HLA⁻DR⁺CD8⁺ T cell as a biomarker for aGVHD. The present application has unexpectedly found that a proportion of the CD38⁺HLA⁻DR⁺CD8⁺ T cell in peripheral blood of a patient with aGVHD is significantly greater than that in peripheral blood of a healthy donor and a patient without aGVHD after transplantation. The receiver operating characteristic (ROC) curve analysis shows that the CD38⁺HLA⁻DR⁺CD8⁺ T cell has an area under the ROC curve (AUC) of 0.84, with a sensitivity of 80.8%, and a specificity of 75.0% when used in early diagnosis of aGVHD. Results indicate that the CD38⁺HLA⁻DR⁺CD8⁺ T cell is a marker for early diagnosis of aGVHD with a prominent diagnostic effect. The marker can guide the clinical medication.

### CROSS-REFERENCE TO RELATED APPLICATIONS

The present application is a continuation-in-part application of PCT application No. PCT/CN2023/116933 filed on Sep. 5, 2023, which claims the benefit of Chinese Patent Application No. 202211078691.8 filed on Sep. 5, 2022. The contents of all of the aforementioned applications are incorporated by reference herein in their entirety.

### TECHNICAL FIELD

The present application belongs to the technical field of medical examination, and specifically relates to a use of a CD38⁺HLA−DR⁺CD8⁺ T cell in early diagnosis of acute graft-versus-host disease (aGVHD). In particular, the present application relates to a test kit for graft-versus-host disease (GVHD) and a method for selecting a detection reagent for GVHD.

### BACKGROUND

GVHD is a systemic disease involving multi-system lesions (including skin, esophagus, gastrointestinal tract, liver, etc.) that occurs after allogeneic hematopoietic stem cell transplantation. GVHD is a specific immune phenomenon resulting from immune responses of immunocompetent cells in the graft tissue to an immunosuppressed host and histoincompatibility antigen-containing tissues. GVHD can be divided into aGVHD and chronic graft-versus-host disease (cGVHD). aGVHD is the leading cause of non-relapse death after allogeneic hematopoietic stem cell transplantation. aGVHD usually occurs within 3 months after allogeneic hematopoietic stem cell transplantation. The immune intolerance of donor T cells to genetically identified proteins on host cells is the essence of GVHD.

aGVHD after allogeneic hematopoietic stem cell transplantation can be divided into the following three stages based on its pathogenesis: 1. Pretreatment stage: The chemoradiotherapy as pretreatment leads to tissue damage, release of inflammatory factors, and antigen exposure. 2.

Induction and proliferation stage of donor T cells: Donor T cells recognize and activate antigens of a recipient, donor T cells and antigens stimulate each other, and donor T cells proliferate excessively. 3. Effective stage: Among donor T cells, activated CD4⁺ cells release cytokines and activated CD8⁺ cells play a cytotoxic role, so as to damage non-hematopoietic tissues of a recipient and cause various clinical manifestations of aGVHD. The clinical diagnosis of aGVHD currently focuses on the effective stage. The current diagnosis of aGVHD is conducted mainly based on clinical manifestations of non-hematopoietic tissue injuries to initiate the treatment, which has high specificity but low sensitivity. The early diagnosis and early treatment of aGVHD are the keys for the successful diagnosis and treatment of aGVHD. The inventors of the present application try to make the early diagnosis and early treatment of aGVHD at the T cell overactivation stage (stage 2).

The major methods for reducing the aGVHD-related morbidity and mortality focus on the prediction of GVHD before clinical manifestations occur and the determination of the optimal time to measure predictors. As a result, there is an opportunity to preemptively stop the progression of GVHD and gain new insights into the pathophysiology of GVHD. In addition, aGVHD risk stratification protocols can be well tested to guide a risk-adapted therapy (by selecting a prevention plan or preemptively conducting an intervention), so as to improve the outcome after allogeneic hematopoietic stem cell transplantation.

The current diagnosis of aGVHD mostly requires the patient to develop pathological changes such as rash and diarrhea, which is not conducive to the timely drug intervention. Therefore, the early diagnosis of aGVHD is particularly important and can guide the clinical medication timely.

CD38⁺HLA⁻DR⁺CD8⁺ T cells are a group of activated T cells. It has been confirmed through research that CD38⁺HLA⁻DR⁺CD8⁺ T cells can serve as a diagnostic marker to distinguish hemophagocytic lymphohistiocytosis from early sepsis. However, the relationship between CD38⁺HLA⁻DR⁺CD8⁺ T cells and aGVHD has not been studied.

### SUMMARY

In view of this, an objective of the present application is to provide a detection reagent for aGVHD, which involves a use of a CD38⁺HLA−DR⁺CD8⁺ T cell in early diagnosis of aGVHD. The detection reagent can allow the detection of aGVHD with high sensitivity and high specificity. Specifically, the detection reagent can allow a detection sensitivity as high as 80.8% and a detection specificity as high as 75.0%.

To allow the above objective, the following specific technical solutions are adopted:

In an aspect, the present application provides a method for selecting a detection reagent for GVHD, including:
providing at least one detection reagent;
detecting proportions of a CD38⁺HLA⁻DR⁺CD8⁺ T cell in biological samples from subjects with the at least one detection reagent;
comparing proportions of the CD38⁺HLA⁻DR⁺CD8⁺ T cell in biological samples from a healthy subject, a subject without the GVHD after transplantation, and a subject with the GVHD;
selecting a detection reagent that is able to detect that a proportion of the CD38⁺HLA⁻DR⁺CD8⁺ T cell in the biological sample from the subject with the GVHD is greater than a proportion of the CD38⁺HLA⁻DR⁺CD8⁺ T cell in the biological samples from the healthy subject and a proportion of the CD38⁺HLA⁻DR⁺CD8⁺ T cell in the subject without the GVHD after the transplantation; and taking the selected detection reagent as the detection reagent for GVHD.

Preferably, the at least one detection reagent refers to 2, 3, 4, 5, 6, 8, 10, or more detection reagents.

In some embodiments, the biological samples are peripheral blood.

In some embodiments, the GVHD is aGVHD.

In some embodiments, the at least one detection reagent is a flow cytometry detection reagent.

In some embodiments, the at least one detection reagent is a flow cytometry detection antibody.

In some embodiments, the flow cytometry detection antibody includes an anti-CD38 fluorescent antibody, an anti-HLA⁻DR fluorescent antibody, and an anti-CD8 fluorescent antibody.

In some embodiments, the subjects are humans.

In some embodiments, the subjects are rats or mice.

The present application also provides a method for diagnosing GVHD, including: detecting a content of a CD38⁺HLA⁻DR⁺CD8⁺ T cell in a biological sample from a host, where the CD38⁺HLA⁻DR⁺CD8⁺ T cell is adopted as a diagnostic marker for GVHD.

In some embodiments, the method includes: detecting proportions of the CD38⁺HLA⁻DR⁺CD8⁺ T cell in biological samples from subjects; and comparing proportions of the CD38⁺HLA⁻DR⁺CD8⁺ T cell in biological samples from a healthy subject, a subject without the GVHD after transplantation, and a subject to be diagnosed, respectively, and when a proportion of the CD38⁺HLA⁻DR⁺CD8⁺ T cell in the biological sample from the subject to be diagnosed is greater than proportions of the CD38⁺HLA⁻DR⁺CD8⁺ T cell in the biological samples from the healthy subject and the subject without the GVHD after the transplantation, determining that the subject to be diagnosed has the GVHD.

In some embodiments, a reagent for detecting a proportion of the CD38⁺HLA⁻DR⁺CD8⁺ T cell is a flow cytometry detection reagent.

In some embodiments, the flow cytometry detection reagent includes an anti-CD38 fluorescent antibody, an anti-HLA⁻DR fluorescent antibody, and an anti-CD8 fluorescent antibody.

In some embodiments, the host is a human or an animal, such as a rat or a mouse.

In some embodiments, the biological samples are peripheral blood.

The present application also provides a test kit for GVHD, including a reagent for detecting a content of a CD38⁺HLA⁻DR⁺CD8⁺ T cell in a biological sample.

In some embodiments, the reagent for detecting a content of the CD38⁺HLA⁻DR⁺CD8⁺ T cell in a biological sample includes an anti-CD38 fluorescent antibody, an anti-HLA⁻DR fluorescent antibody, and an anti-CD8 fluorescent antibody.

In some embodiments, the test kit further includes a reagent for isolating peripheral blood mononuclear cells (PBMCs).

In some embodiments, the test kit further includes a red blood cell lysis buffer and PBS.

The CD38⁺HLA⁻DR⁺CD8⁺ T cell mentioned above refers to a CD38-positive, HLA⁻DR-positive, and CD8-positive T cell.

In the present application, according to the analysis of peripheral blood samples from patients with GVHD, a proportion of the CD38⁺HLA⁻DR⁺CD8⁺ T cell in peripheral blood from a patient with GVHD is significantly greater than proportions of the CD38⁺HLA⁻DR⁺CD8⁺ T cell in peripheral blood from a healthy volunteer and a patient without GVHD after transplantation. According to the receiver operating characteristic (ROC) curve analysis, the CD38⁺HLA⁻DR⁺CD8⁺ T cell has an area under the ROC curve (AUC) of 0.84 (P<0.0001) when used in early diagnosis of aGVHD. The above results indicate that the CD38⁺HLA⁻DR⁺CD8⁺ T cell is a marker for early diagnosis of aGVHD with a prominent diagnostic effect, which has a high clinical application value. The marker can guide the clinical medication timely and alleviate the burden and pain for a patient. Therefore, the detection reagent for the CD38⁺HLA⁻DR⁺CD8⁺ T cell can be used as a reagent for early diagnosis of aGVHD, and can be used to prepare a kit for early diagnosis of aGVHD.

### BRIEF DESCRIPTION OF THE DRAWINGS

FIG. 1 shows flow cytometry analysis results of representative samples, where forward scatter (FSC) refers to a scattered light signal acquired forward, and FSC can reflect a size of cells; side scatter (SSC) refers to a scattered light signal acquired laterally, and an intensity of SSC is related to fine structures and granular properties inside cells; through the FSC/SSC two-parameter assay, human peripheral blood leukocytes are divided into the following three subsets: lymphocytes, monocytes, and granulocytes; gating is conducted within a lymphocyte population, and the adhesions are further removed through a forward scatter area (FSC-A) and a forward scatter height (FSC-H); CD3⁺ cells are then selected within the lymphocyte population, and CD3 is a common surface marker for T cells and can mark all T cells; further, helper T cells (CD3⁺CD4⁺) and killer T cells (CD3⁺CD8⁺) are selected from the CD3⁺ cells; and finally, a cell subset co-expressing CD38 and HLA⁻DR is selected from the CD3⁺CD8⁺ T cells, namely, CD38⁺HLA⁻DR⁺CD8⁺ T cells;

FIG. 2A is a statistical graph of flow cytometry analysis results, where HD represents a healthy volunteer, non-aGVHD represents a patient without GVHD after transplantation, and aGVHD represents a patient with aGVHD;

FIG. 2B shows an ROC curve when the CD38⁺HLA⁻DR⁺CD8⁺ T cell is used for diagnosis of aGVHD, where non-aGVHD represents a patient without aGVHD after transplantation and aGVHD represents a patient with aGVHD;

FIG. 3A and FIG. 3B show the weekly kinetic monitoring of activated CD8⁺ T cells in four patient groups, where FIG. 3A is for non-aGVHD and FIG. 3B is for aGVHD; and according to the comparison between FIG. 3A and FIG. 3B, a patient with an activated CD8⁺ T cell level above a cutoff value (36.6%) within 21 d post-transplantation in the absence of cytomegalovirus (CMV) infection develops aGVHD;

FIG. 4A shows line charts illustrating changes in proportions of activated T in aGVHD patients, where proportions of activated T in peripheral blood of aGVHD patients before a glucocorticoid (GC) treatment (“Before GC”), after a GC treatment (“After GC”), after a hormone-insensitive patient is administered with basiliximab (BX) the first time (“After First BX”), and after the hormone-insensitive patient is administered with BX the third time (“After Third BX”) are dynamically detected, and a change in a proportion of activated T of each aGVHD patient is plotted as a line chart shown in FIG. 4A;

FIG. 4B shows aGVHD scores assessed for each patient and each time point at which each sample is collected in FIG. 4A, where in summary, the international bone marrow transplant registry (IBMTR) aGVHD scores show a parallel decrease with the percentages of activated CD8⁺ T cells during effective treatment; and

FIG. 4C shows detection results of proportions of activated CD8⁺ T cells in peripheral blood of patients at four time points for therapeutic efficacy assessment (complete remission (CR), partial remission (PR), no response (NR), and progression (PD)), where according to the comparison between the aGVHD grading and overall indexing and the initial aGVHD status for each target organ, a patient may undergo CR, PR, NR, and PD, As shown in FIG. 4C, patients who achieved a complete or partial response (CR or PR) exhibited a significant decline in the proportions of activated CD8⁺ T cells below the predetermined cutoff value. Conversely, patients showing no response or progressive disease (NR or PD) maintained activated CD8⁺ T cell levels above the cutoff value. The result indicates that activated CD8⁺ T cells can be used as an effective indicator to assess a therapeutic effect.

### DETAILED DESCRIPTION

In the following examples of the present application, the experimental methods for which specific conditions are not stated are generally conducted according to the conventional conditions or according to the conditions recommended by manufacturers. Various common chemical reagents adopted in the examples are commercially available products.

Unless otherwise defined, all technical and scientific terms used in the present application have the same meaning as commonly understood by those skilled in the technical field of the present application. The terms used in the specification of the present application are provided to merely describe specific examples, rather than limit the present application.

The present application is described below with reference to specific examples.

### Example 1

In this example, contents of CD38⁺HLA⁻DR⁺CD8⁺ T cells in peripheral blood samples from healthy volunteers, patients without GVHD after transplantation, and patients with aGVHD were detected by flow cytometry, which was specifically as follows:

### Experimental Method

### 1. Sample Information

Inclusion Criteria for the Patients with aGVHD:

(1) The patients received allogeneic hematopoietic stem cell transplantation.

(2) The diagnosis for the patients was mainly based on clinical symptoms involving the skin, gastrointestinal tract, and liver and laboratory results. The pathologic biopsy was required for confirmation if necessary. According to the IBMTR grading system, clinical diagnosis results for aGVHD could be divided into grades I to IV. A written informed consent form was signed by a subject or a legally acceptable representative of the subject.

Inclusion Criteria for the Patients without GVHD after Transplantation (Non-GVHD):

(1) The patients received allogeneic hematopoietic stem cell transplantation.

(2) After transplantation and before a sample of a subject was collected, the above clinical and laboratory criteria for aGVHD were not met. The grades I to IV for aGVHD in the IBMTR grading system were not reached.

A written informed consent form was signed by a subject or a legally acceptable representative of the subject.

### Inclusion Criteria for the Healthy Volunteers (HD)

(1) There were no systemic diseases, including: uncontrolled hypertension, unstable angina pectoris, angina pectoris that began within the last 3 months, congestive heart failure (greater than or equal to the grade I of the New York Heart Association (NYHA)), myocardial infarction within half a year, severe arrhythmia requiring a drug therapy, and liver, kidney, or metabolic diseases.

(2) There were no infections or mental diseases.

A written informed consent form was signed by a subject or a legally acceptable representative of the subject.

According to the above inclusion criteria: A total of 46 cases were included in the aGVHD group, including 28 males and 18 females, with an average age of 27 years. A total of 65 cases were included in the Non-GVHD group, including 41 males and 24 females, with an average age of 32 years. A total of 18 cases were included in the HD group, including 11 males and 7 females. The statistical analysis for clinical information of samples was shown in Table 1, and there was no statistically significant difference in clinical information such as age and gender.

TABLE 1

| Variables | Non-aGVHD | aGVHD | p |
|---|---|---|---|
| Days from HSCT to | | | |
| sample colletion | | | |
| Mean (SD) | 50.9 (19.3) | 45.4 (16.6) | 0.108 |
| Median [Min, Max] | 52.0 [13.0, 95.0] | 42.0 [20.0, 79.0] | |
| Disease diagnosis | | | |
| ALL | 4 (6.2%) | 0 (0%) | 0.219 |
| AML | 7 (10.8%) | 6 (13.0%) | |
| MDS | 3 (4.6%) | 5 (10.9%) | |
| SAA | 51 (78.5%) | 35 (76.1%) | |
| Age of Recipient | | | |
| Mean (SD) | 32.5 (11.1) | 27.3 (13.1) | 0.0295 |
| Median [Min, Max] | 32.0 [14.0, 59.0] | 26.5 [8.00, 60.0] | |
| Age of donor | | | |
| Mean (SD) | 29.8 (10.6) | 32.0 (11.3) | 0.289 |
| Median [Min, Max] | 29.0 [10.0, 57.0] | 31.5 [9.00, 55.0] | |
| Gender of Recipient Donor | | | |
| Female to Female | 12 (18.5%) | 6 (13.0%) | 0.289 |
| Female to Male | 24 (36.9%) | 11 (23.9%) | |
| Male to Female | 12 (18.5%) | 13 (28.3%) | |
| Male to Male | 17 (26.2%) | 16 (34.8%) | |
| Condition regimen | | | |
| BuCY + ATG | 23 (35.4%) | 12 (26.1%) | 0.128 |
| DAC + BF + ATG | 12 (18.5%) | 10 (21.7%) | |
| FCA | 10 (15.4%) | 2 (4.3%) | |
| PTCY | 19 (29.2%) | 22 (47.8%) | |
| TBF + ATG | 1 (1.5%) | 0 (0%) | |
| Stem cell source | | | |
| BM + PBSC | 46 (70.8%) | 34 (73.9%) | 0.882 |
| PBSC | 19 (29.2%) | 12 (26.1%) | |
| Donor type | | | |
| HID | 25 (38.5%) | 28 (60.9%) | 0.055 |
| MSD | 30 (46.2%) | 12 (26.1%) | |
| URD | 10 (15.4%) | 6 (13.0%) | |
| ATG dose | | | |
| Mean (SD) | 7.52 (1.78) | 6.96 (1.56) | 0.0784 |
| Median [Min, Max] | 6.00 [6.00, 10.0] | 6.00 [5.00, 10.0] | |

### 2. Flow Cytometry Detection

Peripheral blood samples of each group were collected with EDTA-containing anticoagulant blood collection tubes, and then PBMCs were isolated specifically as follows: PBS was added to a peripheral blood sample with a volume of the PBS 3 times a volume of the peripheral blood sample, and thorough mixing was conducted to obtain a diluted blood sample. A lymphocyte isolation solution was added to a 50 mL sterile centrifuge tube in an equal volume to the diluted blood sample. The diluted blood sample was slowly added along a wall of the 50 mL sterile centrifuge tube to a top of the lymphocyte isolation solution by a pipette. Then the 50 mL sterile centrifuge tube was centrifuged at 18° C. and 600 g (an ascending speed: 5, and a descending speed: 0) for 22 min. After the centrifugation was completed, a middle buffy-coat layer was carefully pipetted and placed in a clean 15 mL centrifuge tube, then 10 mL of pre-cooled PBS was added, and thorough mixing was conducted. The 15 mL centrifuge tube was centrifuged at 2,000 rpm/min and room temperature for 5 min to obtain a first supernatant and a first cell pellet, and the first supernatant was discarded. 3 mL of a red blood cell lysis buffer (ACK) was added to the first cell pellet, red blood cell lysis was conducted at room temperature for 5 min, and then 10 mL of pre-cooled PBS was added to stop the red blood cell lysis. The 15 mL centrifuge tube was centrifuged at 2,000 rpm/min and room temperature for 5 min to obtain a second supernatant and a second cell pellet, and the second supernatant was discarded. The second cell pellet was resuspended with 5 mL of PBS to obtain a single cell suspension of PBMCs, and the single cell suspension of PBMCs was counted and then tested by flow cytometry.

An antibody combination adopted in the flow cytometry detection was as follows: PE Anti-Human CD38 (BIOLEGEND (Beijing), Item No.: 356604), APC-Cyanine7 Anti-Human HLA⁻DR (TONBO BIOSCIENCES, Item No.: 25-9952-T100), PerCP/Cyanine5.5 anti-human CD8 (BIOLEGEND (Beijing), Item No.: 301032), and PE-Cyanine7 Anti-Human CD3 (TONBO BIOSCIENCES, Item No.: 60-0037-T100). A detection method was as follows:

(1) 1 million of PBMCs were taken and added to a flow cytometry tube, then 5 mL of PBS was added, and thorough mixing was conducted. The flow cytometry tube was centrifuged at 2,000 rpm/min for 5 min to obtain a first supernatant and a first cell pellet, and the first supernatant was discarded.

(2) According to antibody instructions, the anti-CD38, anti-HLA⁻DR, anti-CD3, and anti-CD8 antibodies were added to PBS in a volume ratio of 1:200 to obtain an antibody dilution. 100 μL of the antibody dilution was added to the first cell pellet for staining. The flow cytometry tube was shaken on a shaker to allow thorough mixing, and then placed in a 4° C. refrigerator to allow staining in the dark for 30 min.

(3) After the staining was completed, 4 mL of PBS was added to the flow cytometry tube. The flow cytometry tube was centrifuged at 2,000 rpm/min for 5 min to obtain a second supernatant and a second cell pellet, and the second supernatant was discarded. The second cell pellet was resuspended with 300 μL of PBS to obtain a sample ready for testing.

(4) The sample was tested by a flow cytometer and analyzed with the FlowJo10 software.

### 3. Statistical Analysis

The GraphPad 9.2.0 was adopted to calculate a median and a P value for data of each group, and P<0.05 was regarded as a statistically significant difference.

### Experimental Results

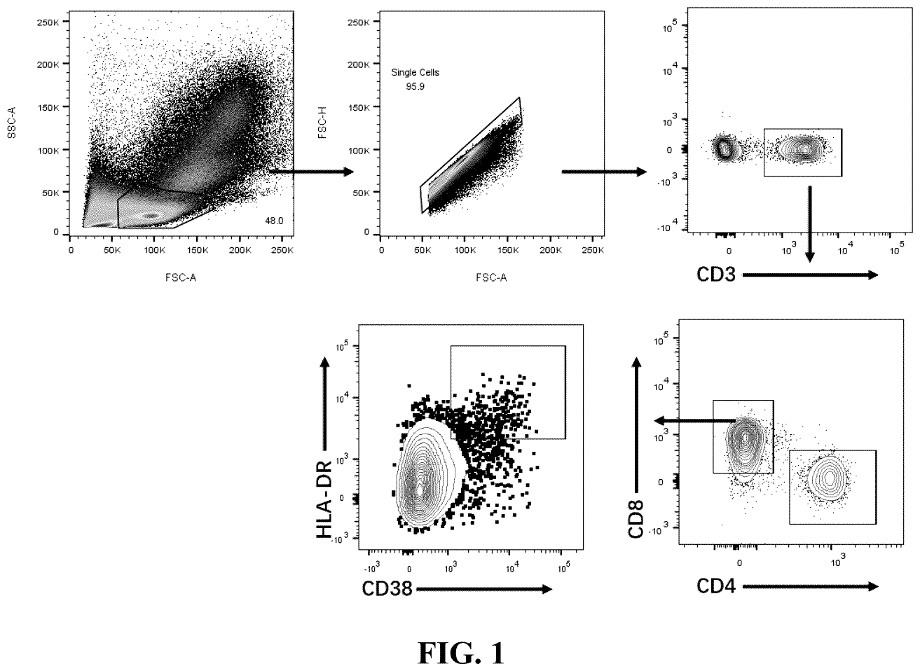

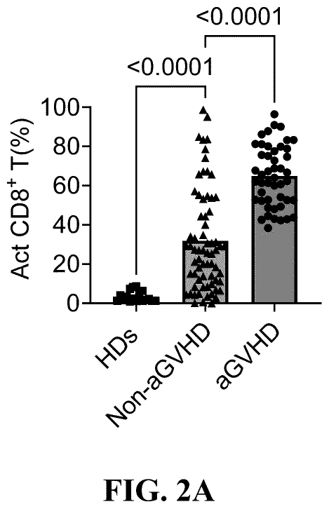

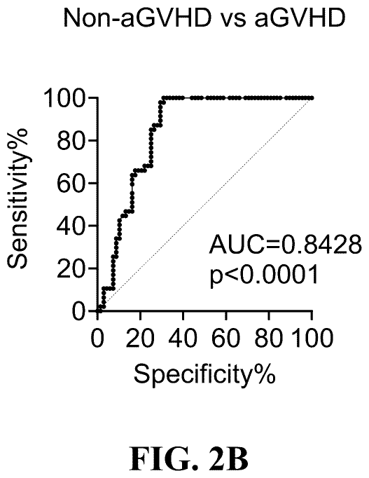

A flow cytometry analysis process was shown in FIG. 1. A proportion of CD38⁺HLA⁻DR⁺CD8⁺ T cells in each sample was obtained according to the analysis method shown in FIG. 1, and the statistical analysis was conducted. FIG. 1 also showed a gating strategy for activated CD8⁺ T lymphocytes in flow cytometry. Lymphocyte populations distinguished by FSC and SSC were circled by rectangles. Then, in this figure, a gate was set to select single cells (excluding debris and double-cell aggregates). CD3 T cells, CD8 T cells, and CD4 T cells were marked with makers CD3, CD8, and CD4, respectively, and then CD38⁺HLA⁻DR⁺CD8⁺ T lymphocytes were marked with anti-CD38 and anti-HLA⁻DR antibodies. FIG. 2A showed a statistical graph of flow cytometry analysis results. As shown in FIG. 2A, compared with the healthy volunteers (HDs) and the patients without GVHD after transplantation (non-aGVHD), a proportion of the CD38⁺HLA⁻DR⁺CD8⁺ T cell in peripheral blood of the patients with aGVHD was significantly increased, indicating that the CD38⁺HLA⁻DR⁺CD8⁺ T cell could be used as a potential diagnostic markers for aGVHD. As a result, the ROC curve analysis was conducted in the present application. ROC curve analysis results showed that the CD38⁺HLA⁻DR⁺CD8⁺ T cell had an AUC of 0.84, a sensitivity of 80.8%, and a specificity of 75.0% when used in early diagnosis of aGVHD (FIG. 2B).

The above results indicate that the CD38⁺HLA⁻DR⁺CD8⁺ T cell is a marker for early diagnosis of aGVHD with a prominent diagnostic effect and a high accuracy, the marker has a high clinical application value, and the marker can guide the clinical medication timely and reduce the aGVHD-related morbidity and mortality.

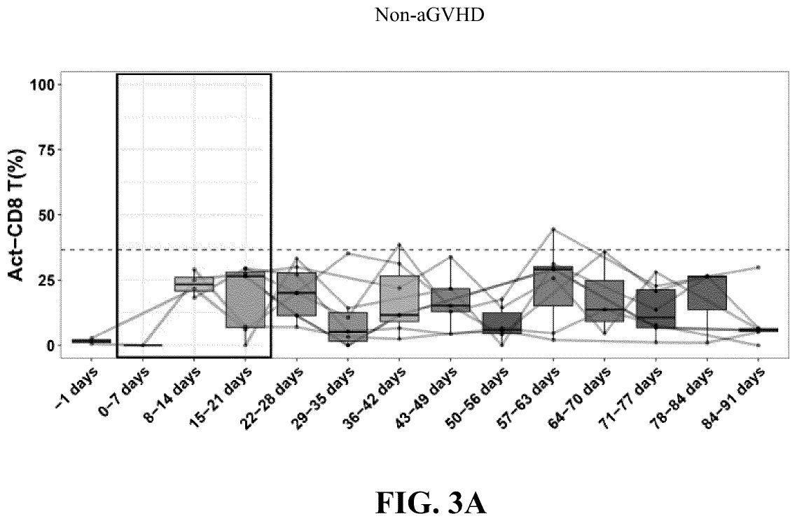

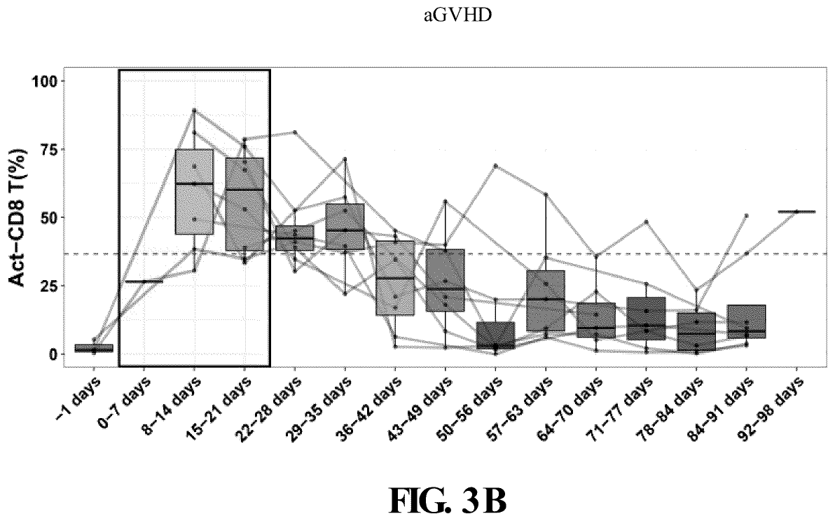

The dynamic follow-up was conducted once a week for activated T cell levels in patients after transplantation (FIG. 3A and FIG. 3B). The follow-up began on day 7 after transplantation and ended on day 91 to day 98 after transplantation. The isolation method for activated T cells, the experimental materials and reagents, and the experimental steps were detailed in the flow cytometry detection section. FIG. 3A showed a dynamic change of activated T cells in the patients without aGVHD (non-aGVHD) within 91 d after transplantation. FIG. 3B showed a dynamic change of activated T cells in the patients with aGVHD within 98 d after transplantation. Each broken line in the above figures showed a dynamic change of activated T cells in a same patient after transplantation. The green box in FIG. 3A (namely, the largest rectangular box in this figure through which the dotted line passes) showed that, when a proportion of activated CD8⁺ T cells was lower than a cut-off value in the first three weeks, aGVHD was not developed subsequently. In contrast, the green box in FIG. 3B (namely, the largest rectangular box in this figure through which the dotted line passes) showed that, when a proportion of activated CD8⁺ T cells was greater than the cut-off value in the first three weeks, aGVHD was developed subsequently. According to the comparison between FIG. 3A and FIG. 3B, all patients with an activated CD8⁺ T cell level greater than the cut-off value within 21 d after transplantation developed aGVHD.

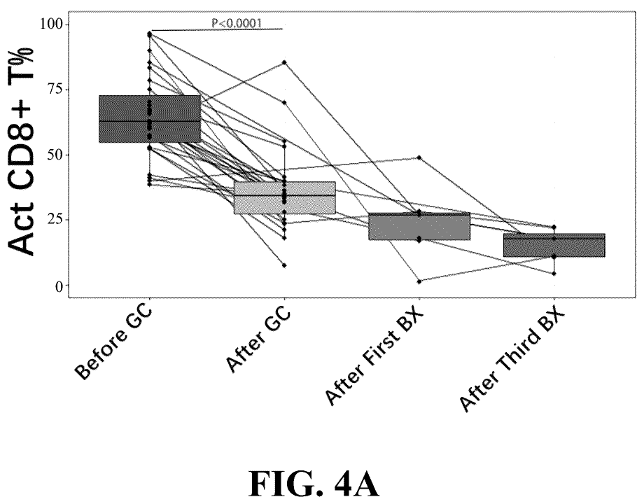

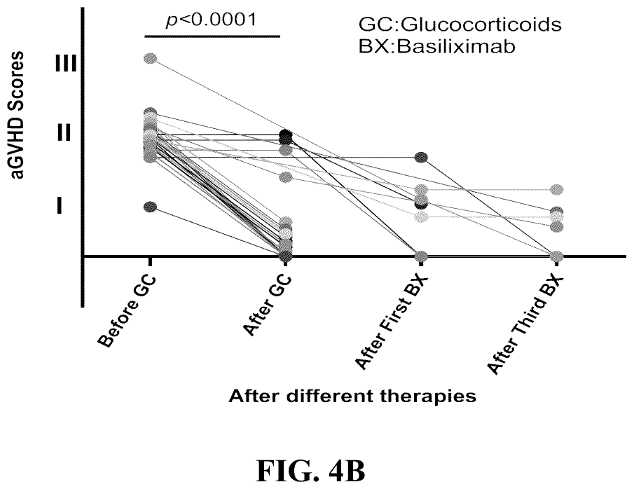

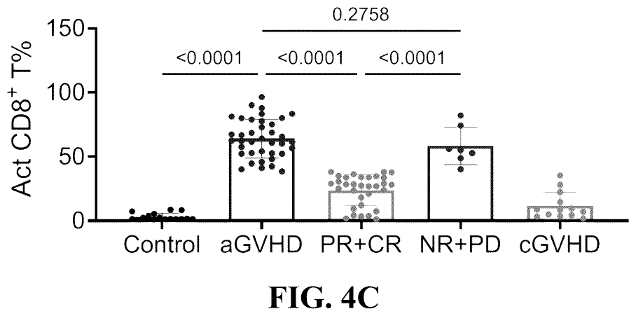

According to the Chinese Expert Consensus of Allogeneic Hematopoietic Stem Cell Transplantation for Treating Hematological Diseases (III)-Acute Graft-Versus-Host Disease (aGVHD) (2020 Edition)^{[1]}, the therapeutic efficacy assessment was conducted through the comparison between the aGVHD grading and overall indexing and the initial aGVHD status for each target organ, and a patient may undergo CR, PR, NR, and PD. After the initial GC treatment and the subsequent BX (an anti-CD25 monoclonal antibody) treatment, as shown in FIG. 4A, an activated CD8⁺ T cell level was decreased significantly. The statistical analysis of treatment outcomes showed that a proportion of activated CD8⁺ T cells in a patient undergoing CR or PR was significantly lower than the predetermined cut-off value. In contrast, an activated CD8⁺ T cell level in a patient undergoing NR or PD was greater than the cut-off value (FIG. 4C). The aGVHD scores according to the IBMTR scoring system were consistent with the observed trend in a percentage of activated CD8⁺ T cells, indicating that the indicator is reliable in the assessment of a therapeutic effect (FIG. 4A and FIG. 4B). In conclusion, this systematic clinical study confirmed that the activated CD8⁺ T cell could serve as a marker for early prediction of the occurrence of aGVHD and could serve as an effective indicator to assess a therapeutic effect.

The technical characteristics of the above embodiments can be arbitrarily combined. For brevity of description, not all possible combinations of the technical characteristics of the above embodiments are described. However, these combinations of the technical characteristics should be construed as falling within the scope defined by the specification as long as there is no contradiction among the combinations.

The above embodiments are merely illustrative of several implementations of the present disclosure, and the description thereof is specific and detailed, but should not be construed as a limitation to the patent scope of the present disclosure. It should be noted that those of ordinary skill in the art can further make several variations and improvements without departing from the concept of the present application, and these variations and improvements all fall within the protection scope of the present application. Therefore, the protection scope of the present application should be subject to the protection scope defined by the claims.

### CITED REFERENCES

1. [Chinese consensus of allogeneic hematopoietic stem cell transplantation for hematological disease (III)-acute graft-versus-host disease (2020)]. Zhonghua Xue Ye Xue Za Zhi, 2020. 41 (7): p. 529-536.

1. A method for selecting a detection reagent for graft-versus-host disease (GVHD), comprising:
providing at least one detection reagent;
detecting proportions of a CD38⁺HLA⁻DR⁺CD8⁺ T cell in biological samples from subjects with the at least one detection reagent;
comparing proportions of the CD38⁺HLA⁻DR⁺CD8⁺ T cell in biological samples from a healthy subject, a subject without the GVHD after transplantation, and a subject with the GVHD;
selecting a detection reagent that is able to detect that a proportion of the CD38⁺HLA⁻DR⁺CD8⁺ T cell in the biological sample from the subject with the GVHD is greater than a proportion of the CD38⁺HLA⁻DR⁺CD8⁺ T cell in the biological samples from the healthy subject and a proportion of the CD38⁺HLA⁻DR⁺CD8⁺ T cell in the subject without the GVHD after the transplantation; and
taking the selected detection reagent as the detection reagent for GVHD.

2. The method according to claim 1, wherein the biological samples comprise peripheral blood.

3. The method according to claim 1, wherein the GVHD comprises acute graft-versus-host disease (aGVHD).

4. The method according to claim 1, wherein the at least one detection reagent comprises a flow cytometry detection reagent.

5. The method according to claim 4, wherein the at least one detection reagent comprises a flow cytometry detection antibody.

6. The method according to claim 5, wherein the flow cytometry detection antibody comprises an anti-CD38 fluorescent antibody, an anti-HLA⁻DR fluorescent antibody, and an anti-CD8 fluorescent antibody.

7. The method according to claim 1, wherein the subjects comprise humans.

8. The method according to claim 1, wherein the subjects comprise rats or mice.

9. A test kit for GVHD, comprising a reagent for detecting a content of a CD38⁺HLA⁻DR⁺CD8⁺ T cell in a biological sample.

10. The test kit according to claim 9, wherein the reagent for detecting a content of the CD38⁺HLA⁻DR⁺CD8⁺ T cell in a biological sample comprises an anti-CD38 fluorescent antibody, an anti-HLA⁻DR fluorescent antibody, and an anti-CD8 fluorescent antibody.

11. The test kit according to claim 9, wherein the test kit further comprises a reagent for isolating peripheral blood mononuclear cells (PBMCs), a red blood cell lysis buffer, and phosphate-buffered saline (PBS).

12. The test kit according to claim 9, wherein the GVHD comprises aGVHD.

13. A method for diagnosing GVHD, comprising:
detecting proportions of a CD38⁺HLA⁻DR⁺CD8⁺ T cell in biological samples from subjects; and
comparing proportions of the CD38⁺HLA⁻DR⁺CD8⁺ T cell in biological samples from a healthy subject, a subject without the GVHD after transplantation, and a subject to be diagnosed, and
when a proportion of the CD38⁺HLA⁻DR⁺CD8⁺ T cell in the biological sample from the subject to be diagnosed is greater than proportions of the CD38⁺HLA⁻DR⁺CD8⁺ T cell in the biological samples from the healthy subject and the subject without the GVHD after the transplantation, determining that the subject to be diagnosed has the GVHD.

14. The method according to claim 13, wherein a reagent for detecting a proportion of the CD38⁺HLA⁻DR⁺CD8⁺ T cell comprises a flow cytometry detection reagent.

15. The method according to claim 14, wherein the flow cytometry detection reagent comprises an anti-CD38 fluorescent antibody, an anti-HLA⁻DR fluorescent antibody, and an anti-CD8 fluorescent antibody.
